(19) United States
(12) Patent Application Publication
(10) Pub. No.: US 2014/0257080 A1
(43) Pub. Date: Sep. 11, 2014

(54) SYSTEM FOR ULTRASOUND IMAGE GUIDED PROCEDURE

(71) Applicant: EZONO AG, Jena (DE)

(72) Inventors: Allan Dunbar, Jena (DE); Eliseo Sobrino, Jena (DE)

(73) Assignee: eZono AG, Jena (DE)

(21) Appl. No.: 13/785,951

(22) Filed: Mar. 5, 2013

(51) Int. Cl. A61B 8/00; A61B 5/06; A61M 25/01; A61B 5/00; A61B 17/34; A61M 25/09; A61B 5/05; A61B 8/08

(52) U.S. Cl. CPC A61B 8/4416; A61B 5/05; A61B 5/062; A61B 8/0841; A61B 8/52; A61B 5/72; A61B 17/34; A61M 25/09; A61M 25/0108; A61M 25/0127
USPC 600/409

(57) ABSTRACT

A system and method for ultrasound image guided surgical procedures such as needling or catheterisation, in which an ultrasound transducer is provided with a magnetometric detector for detecting the magnetic field emanating from a magnetised tissue-penetrating medical tool such as a needle or catheter. The detection of the magnetic field allows the position of the tool to be tracked magnetically and the position can be displayed on the ultrasound image. The position of the tool is determined from the magnetic field measurements by use of a look-up table of magnetic field values for the field emanating from the tissue-penetrating medical tool.

### TECHNICAL FIELD

The present invention relates generally to the field of medical devices and in particular to a system for improving image guided procedures such as needle or catheterisation procedures.

### BACKGROUND AND OVERVIEW

Unless explicitly indicated herein, the materials described in this section are not admitted to be prior art.

There are numerous medical procedures that involve the insertion of a medical tool or instrument, such as a needle, cannula, catheter or stylet, into a subject's body, e.g. minimally-invasive surgical procedures, regional anaesthesia, detection of bio-electrical signals, electrical stimulation for diagnosis or treatment, vascular access, fine needle aspiration, musculoskeletal injections and so on. In such procedures it is generally necessary to guide the medical tool properly to the desired position in the subject's body and it can also be beneficial to monitor or track the medical tool position to ensure that it remains at the desired location. In general it is very difficult for the user to determine the exact position of the tip of the medical tool and thus to be sure whether it is in the desired place, for example adjacent a nerve, or whether it has undesirably penetrated something else, for example a blood vessel.

It has been proposed to use x-ray techniques for needle guidance by providing the clinician with an x-ray image of the needle in the body. However in view of the risks associated with exposure to electromagnetic radiation, it is not possible to provide a continuous guidance during insertion of the medical tool and so a series of snapshots are relied upon, which does not give optimal guidance.

More recently the use of ultrasound imaging to guide needle and catheterisation procedures has been proposed. Ultrasound imaging is advantageous compared to x-ray techniques because of the lack of exposure to electromagnetic radiation, and ultrasound probes are easily manipulable to image many different parts of the body. However ultrasound imaging has two main challenges: firstly that the interpretation of ultrasound images is rather difficult, and secondly that needles do not show-up particularly reliably or visibly in the ultrasound image.

As to the problem of needle visibility, the ultrasound image acquisition plane is thin—of the order of 1 mm thick, and so if the needle is out of that plane it will not be imaged. Further, even when the needle is in the imaging plane, because the echogenicity of standard needles is poor at high angles of incidence, the needle may not be particularly visible. It has been proposed to produce echogenic needles which make the needle more visible to ultrasound imaging devices. However these only help when the needle is well-aligned with the imaging plane. Similarly techniques for image processing and ultrasound beam steering help only when the needle is well-aligned with the imaging plane and do not work well for angles of incidence greater than 45 degrees.

Various needle tracking technologies have been proposed based either on a needle guide fitted to an ultrasound probe, e.g. U.S. Pat. No. 6,690,159 or WO-A-2012/040077, or based on the transmission and reception of electromagnetic information, e.g. US-A-2007-027390) but have functional and accuracy limitations which means that the needle tip position is not exactly known in every clinical circumstance. Typical accuracies are of the order of 2 mm, which can mean the difference between the needle tip being inside or outside a nerve. Further they often require the use of heavily modified or new equipment which is unwelcome to clinicians and to institutions with relatively rigid purchasing regimes.

Most often, therefore, practitioners rely on their skill and experience to judge where the tip of the medical instrument is as it is inserted. They may rely on sound, the touch and feel of the physical resistance to the medical tool and sudden changes in resistance, and changes in resistance to the injection of air or fluids. Developing this level of skill and experience is time-consuming and difficult and as there is an anatomical variation from patient to patient, the procedures inevitably entail some risks.

In summary, although ultrasound guidance has improved some needling procedures, there are still significant difficulties and it cannot be used for many procedures. This is a major barrier to its widespread use, particularly its use by practitioners who are not medical imaging specialists, such as anaesthetists, surgeons, pathologists, emergency physicians etc.

Accordingly, the present invention provides an improved method and system for ultrasound image-guided procedures which combines ultrasound imaging of the subject's internal anatomy with magnetic tracking of the tissue-penetrating medical tool and display of the tracked position on the displayed anatomical image.

The magnetic tracking is achieved by a magnetic position detection system which uses a magnet and a magnetometric detector, one on the tool to be tracked and one on the ultrasound probe, to detect the relative position of the tool and probe. The magnetic field detected by the detector is compared to magnetic field data in a look-up table to find the relative position. The look-up table contains magnetic field data obtained by measuring the field generated by the magnet, optionally also interpolated data.

In a preferred embodiment the magnetic position detection system may comprise a magnetised tissue-penetrating medical tool and a magnetometric detector on the ultrasound probe. This has the advantage that the tissue-penetrating tool is a standard one which has been magnetised, and that a freehand ultrasound transducer may be used.

The tissue-penetrating medical tool may be a needle, catheter, cannula or stylet or the like.

In more detail one aspect of the present invention provides an ultrasound imaging system for image-guided medical procedures, the system comprising: an ultrasound transducer probe for transmitting ultrasound into a subject and receiving ultrasound echoes from the subject and outputting ultrasound echo data; a magnetometric detector attached to the ultrasound transducer probe for detecting a magnetic field emanating from a tissue-penetrating medical tool and outputting measurements of the magnetic field; a data processor adapted to receive the ultrasound echo data and process it to produce an ultrasound image and adapted to receive the magnetic field measurements and process them to determine the position of the tissue-penetrating medical tool relative to the ultrasound transducer probe; a data store storing a look-up table of values of the magnetic field emanating from the tissue-penetrating medical tool; the data processor being adapted to determine the position of the tissue-penetrating medical tool relative to the ultrasound transducer probe by comparing the magnetic field measurements to the values of the magnetic field stored in the look-up table.

One advantage of using a look-up table is that any shape of magnetisable tool can be tracked compared to only modellable shapes, e.g. a needle or elongated shape, with a model-based approach. With the look-up table the shape is not limited to simple geometries. In some cases this may also be an advantage compare to a model-based method as the clinician may be interested in tracking or locating complex-shaped objects entering the body, e.g. a scalpel, or already embedded in the body, e.g. a screw.

Another advantage is that models only describe the real world to a certain accuracy. Imperfections compared to idealized model-based approaches introduce errors in position detection. For example a model-based approach may achieve an accuracy in the range of plus or minus 2 mm (3 sigma) for the tip position. When measuring the field for use in a look-up table, e.g. robotically, the repeatability of one needle path can be 0.1 mm (3 sigma). This indicates that the potential accuracy of the measurement system is much higher than model-based position estimation. Further the look-up table approach allows measurement of the exact field of interest.

The tissue-penetrating medical tool can be a needle, stylet, cannula, catheter or the like. Preferably plural look-up tables are provided for respective different tools. The look-up table preferably stores values of the direction and magnitude of the magnetic field at a plurality of spatial positions around the tool. Preferably the spatial position are in a three dimensional grid array around the tool, for example at a spatial resolution of 1 to 5 mm and extending from the tool by a distance of up to 200 mm, more preferably up to, 150 mm or 100 mm, or 75 mm, or up to 50 mm. The size of the measurement area, i.e. the working range of the system, depends on the strength of the magnetic field compared to the noise level in the sensors. A low noise level means that smaller fields can be measured, so the stronger the field generated by the tool and the lower the noise level in the sensors the greater the range. The field generated by the tool depends on its size and shape—a thicker metallic element such as a screw may be able to generate a stronger field than a thin needle for example.

The magnetometric detector may comprise an array of magnetometers. In that case the look-up table can comprise values of the directions and magnitude of the magnetic field for each sensor position of the array for a plurality of angular orientations of the array at each of the plurality of spatial positions of the ultrasound transducer around the tool. Thus each spatial position of the transducer will be associated with multiple sets of magnetic field values representing the readings from sensors in the array with different angular orientations of the transducer at that position.

The magnetic field measurement may comprise the magnitude in each of three orthogonal directions, with the magnetometric detector detecting the magnitude of the field in three orthogonal directions.

The invention also provides a corresponding method comprising the steps of providing an ultrasound transducer probe for transmitting ultrasound into a subject and receiving ultrasound echoes from the subject and outputting ultrasound echo data; attaching a magnetometric detector to the ultrasound transducer probe for detecting a magnetic field emanating from a tissue-penetrating medical tool and outputting measurements of the magnetic field; providing a data processor adapted to receive the ultrasound echo data and process it to produce an ultrasound image and adapted to receive the magnetic field measurements and process them to determine the position of the tissue-penetrating medical tool relative to the ultrasound transducer probe; providing a data store storing a look-up table of values of the magnetic field emanating from the tissue-penetrating medical tool; wherein the data processor is adapted to determine the position of the tissue-penetrating medical tool relative to the ultrasound transducer probe by comparing the magnetic field measurements to the values of the magnetic field stored in the look-up table.

The values for the look-up table may be obtained by measuring the field emanating from a tissue-penetrating medical tool, preferably using a magnetometric detector corresponding to the one used in the position tracking process. Thus the field may be measured for the look-up table by using a magnetometric detector comprising an array of magnetometers corresponding to the array provided on the ultrasound transducer. The look-up table may store values of direction and magnitude of the field for a plurality of spaced transducer locations around the tool, and preferably for a plurality of angular orientations of the transducer at each position.

The look-up table may be populated not only by direct measurements of the field, but also by values calculated by interpolation of the measurements, this reducing the time needed to measure the magnetic field. Preferably the symmetry of the magnetic field around the tool is used to reduce the number of values stored in the look-up table.

A robotic arrangement for holding the tool 5 and moving a magnetometric detector around the tool to measure the field or vice versa and populate the look-up table may be used. Alternatively the look-up table may be populated by using measurements from a large array of sensors measuring the magnetic field at multiple positions simultaneously.

Thus with the invention the fact that the tissue-penetrating medical tool may have a low echogenecity is overcome by using magnetic position detection, in particular by magnetising the tool and using an array of magnetometers on the ultrasound transducer to detect the field from the magnetised tool. The magnetically detected position and/or track of the tool is then displayed in the ultrasound image.

If the tissue penetrating medical tool is out of the imaging plane of the ultrasound transducer then the processor and display may be adapted to show a position of the tool projected into the ultrasound imaging plane. The fact that it is a projected position can be indicated by visually distinguishing it from an actual position, for example by showing it dotted or in a different colour.

The data processor or processors used in the invention can be adapted to their data processing task by being dedicated signal processors (e.g. effectively hard-wired to the purpose), or based on programmable processors such as ASICs or FPGAs, or firmware or software-controlled general-purpose processors.

The invention therefore makes available to the clinician the image information and the detected position information. The delivery and presentation to the clinician of this information make the surgical procedure much safer. Further, it achieves this without substantial modification of the instruments used by the clinician and thus without needing substantial modification of the surgical procedures.

### BRIEF DESCRIPTION OF THE DRAWINGS

The invention will be further described by way of examples with reference to the accompanying drawings in which:

FIG. 1 is a schematic diagram of a system according to one embodiment of the invention;

FIG. 2 schematically illustrates in block diagram form a magnetometric detector according to one embodiment of the invention; and

FIG. 3 schematically illustrates in block diagram form a base station for the magnetometric detector of FIG. 2; and

FIG. 4 schematically illustrates in block diagram form the data processing and display steps of one embodiment of the invention.

### DETAILED DESCRIPTION

As shown in FIG. 1 the system in this embodiment of the invention comprises an ultrasound imaging system 1 including an ultrasound transducer 2, processor 3 and display 4. The system also comprises a tissue-penetrating medical tool 5 such as a needle, stylet, catheter or cannula.

The invention uses magnetic position detection to track the tissue penetrating tool 5. Thus in this embodiment the tool 5 is magnetised and the ultrasound transducer 2 is provided with a magnetometric detector 12 comprising an array 100 of magnetometers 120. The detector 12 senses the magnetic field from the tool 5, together with the terrestrial magnetic field and any other background magnetic field, and the processor 3 is adapted to determine from the detected field the position and orientation of the tool 5 relative to the transducer 2. This magnetically detected position is then displayed on the display 4 together with the ultrasound image.

The ultrasound system 1 can be a standard two dimensional B-mode ultrasound system with the standard ultrasound probe 2 being modified by the provision of the magnetometric detector 12. The processor 4, which is connected to the ultrasound probe via a cable, drives the ultrasound transducer 2 by sending electrical signals to cause it to generate ultrasound pulses and interpreting the raw data received from the transducer 2, which represents echoes from the subject's body, to assemble it into an image of the patient's tissue.

The magnetometric detector 12 may be detachably attached to the ultrasound transducer 2 and can be battery-powered or powered from the ultrasound system. Preferably positioning elements are provided on the magnetometric detector 12 to ensure that it is always attached in the same well-defined position and orientation. The magnetometric detector 12 is connected by a wireless connection 15 to a base unit 14 which is in wireless or wired (e.g. USB) communication 16 with the ultrasound processor 3 and display 4. The base unit 14 can be integrated with, or some of its functions performed by, the ultrasound processor 3 or the magnetometric detector 12.

As will be explained in more detail below, the base unit 14 receives normalised measurements from magnetometric detector 12 and calculates the position, or optionally the position and orientation, of the medical tool 5. The base unit 14 can also receive additional information such as the state of charge of the magnetometric detector's battery and information can be sent from the base unit 14 to the magnetometric detector 12, such as configuration information. The base unit 14 forwards the results of its calculations, i.e. the position and, optionally, orientation, to the ultrasound image processor 3 for inclusion in the displayed ultrasound image of an image 17 of the tool 5.

Although the use of the base station 14 is advantageous in requiring less modification of the ultrasound system 1, it will be appreciated that it can be integrated into the ultrasound system 1 with the processor 3 taking-over the functions of the processor 180 and the magnetometric detector 12 being in direct communication with the ultrasound system 1 either via wireless link or using the same physical cable as the ultrasound probe 2.

The magnetometric detector 12 and the way in which the position of the magnetised tool 5 compared to the ultrasound probe 2 are calculated will now be explained in more detail.

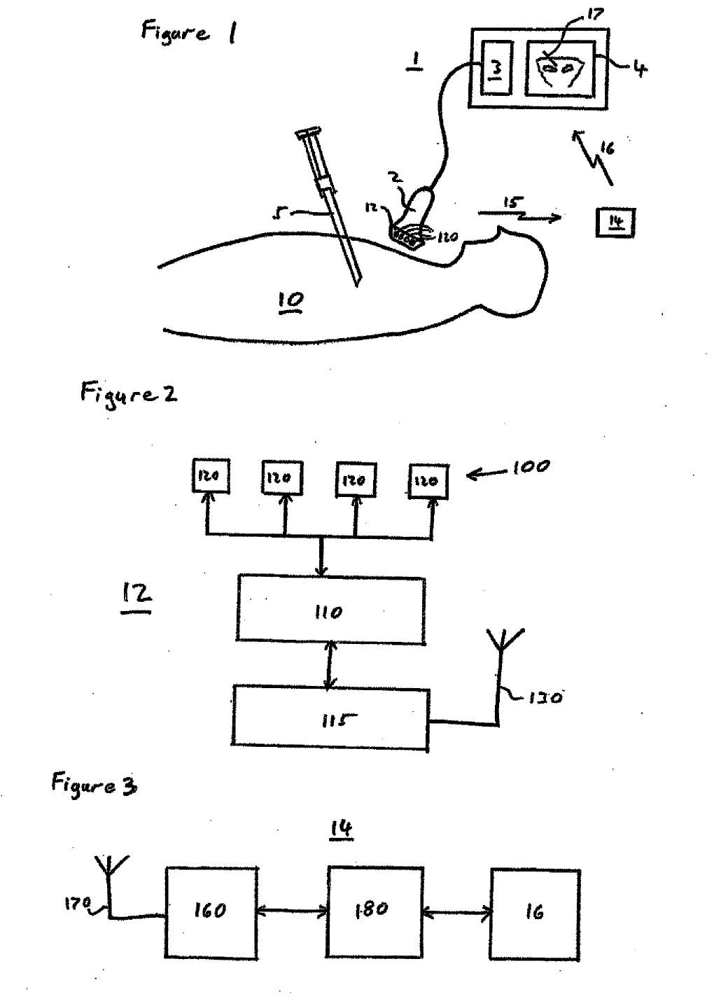

The components of the magnetometric detector 12 are shown schematically in greater detail in the block diagram of FIG. 2. The magnetometric detector 12 comprises an array 100 or two or more (e.g. four) magnetometers 120 whose outputs are sampled by a microprocessor 110. The microprocessor 110 normalizes the measurement results obtained from the magnetometer array 100 and forwards them to a transceiver 115 with an antenna 130 which, in turn transmits the information to the base unit 14. In a modified version of this embodiment, the magnetometric detector 12 is provided with a multiplexer rather than with a microprocessor 110 and the normalization is performed by a processor 180 in the base unit 14.

Each magnetometer 120 in the array 100 of magnetometers measures the components aₖ^{u}, aₖ^{v}, aₖ^{w} (k indicating the respective magnetometer) of the magnetic field at the position of the respective magnetometer 120 in three linearly independent directions. The microprocessor 110 transforms these raw values:

aₖ=(aₖ^{u}, aₖ^{v}, aₖ^{w})

into corresponding normalized values:

bₖ=(bₖ^{x}, bₖ^{y}, bₖ^{z})

in predetermined orthogonal directions of equal gain by multiplying the three values aₖ obtained from the magnetometer with a normalisation matrix Mₖ and adding a normalisation offset vector βₖ:

bₖ=aₖ*Mₖ+βₖ

as will be described in more detail below. The normalisation matrices and the normalisation offset vectors are permanently stored in a memory associated with the microcontroller 110. This same transformation is performed for each of the magnetometers 120 with their respective normalisation matrix and adding a normalisation offset vector such that the result bₖ, for each magnetometer provides the components of the magnetic field in the same orthogonal spatial directions with identical gain. Thus, in a homogenous magnetic field, all magnetometers always provide identical values after normalisation regardless of the strength or orientation of the homogenous magnetic field.

### Normalisation and Offset

All magnetometers should measure equal values when exposed to a homogeneous field. For example, a magnetometer rotated in the homogeneous terrestrial magnetic field should, depending on the orientation of the magnetometer, measure varying strengths of the components of the magnetic field in the three linearly independent directions. The total strength of the field, however, should remain constant regardless of the magnetometer's orientation. Yet, in magnetometers available on the market, gains and offsets differ in each of the three directions. Moreover, the directions oftentimes are not orthogonal to each other. As described for example in U.S. Pat. No. 7,275,008 B2 for a single sensor, if a magnetometer is rotated in a homogeneous and constant magnetic field, the measurements will yield a tilted 3-dimensional ellipsoid. Because the measured field is constant, however, the normalized measurements should lie on a sphere. Preferably, an offset value β and a gain matrix M are introduced to transform the ellipsoid into a sphere.

With a set of sensors, additional steps need to be taken to assure that the measurements of different sensors are identical with each other. To correct this, preferably, set of a gain normalisation matrices Mₖ and normalisation offset vectors βₖ for each position k are determined which transform the magnetometer's raw results aₖ into a normalized result bₖ:

bₖ=aₖ*Mₖ+βₖ

Such a set of gain matrices Mₖ can be obtained by known procedures, for example the iterative calibration scheme described in Dorveaux et. al., “On-the-field Calibration of an Array of Sensors”, 2010 American Control Conference, Baltimore 2010.

By virtue of the defined transformation, bₖ provides the strength of the component of the magnetic field in three orthogonal spatial directions with equal gain. Moreover, it is ensured that these directions are the same for all magnetometers in the magnetometric detector. As a result, in any homogeneous magnetic field, all magnetometers yield essentially identical values.

The normalisation information Mₖ and βₖ for each magnetometer as obtained in the calibration step can be stored either in the magnetometric detector 12 itself or in the base unit 14. Storing the information in the magnetometric detector 12 is preferred as this allows easy exchange of the magnetometric detector 12 without the need to update the information in the base unit. Thus, in a preferred embodiment of the invention, the outputs of the magnetometers of the magnetometric device are sampled and their results are normalised in the magnetometric detector 12. This information, possibly together with other relevant information, is transmitted to the base unit 14 for further analysis.

In another embodiment of the invention, the transformation can be another, more general non-linear transformation bₖ=F(aₖ).

In addition to the above calibration method, another calibration method is applied which employs an inhomogeneous magnetic field to obtain the relative spatial locations of the magnetometric detector's magnetometers. While standard calibration methods utilize a homogenous magnetic field to (a) align the measurement axis of the magnetometers orthogonally, (b) cancel the offset values and (c) adjust to equal gain, it is of further advantage to the described systems that also the precise relative spatial locations of the magnetometers are available. This can be achieved by an additional calibration step in which the magnetometric detector is subjected to a known inhomogeneous magnetic field. Preferably, comparing the obtained measurements at the various positions to the expected field strengths and/or orientations in the assumed locations, and correcting the assumed locations until real measurements and expected measurements are in agreement, allows for the exact calibration of the spatial positions of the sensors.

In a variation of the latter calibration method, an unknown rather than a known homogeneous field is used. The magnetometers are swept through the unknown magnetic field at varying positions, with a fixed orientation. With one of the magnetometers supplying a reference track, the positions of the other magnetometers are adaptively varied in such a way that their measurements align with the measurements of the reference unit. This can be achieved for example by a feedback loop realizing a mechano-magnetic-electronic gradient-descent algorithm. The tracks used in this inhomogeneous field calibration can be composed of just a single point in space.

### Position Detection

The base station 14 shown schematically in greater detail in FIG. 3 receives the normalised positional information from the magnetometric detector 12 through its receiver 160 with antenna 170 and forwards the information to a processor 180. There, the normalized results of the measurements are used to derive the position (or position and orientation) of the tool 5. The calculated position and orientation are transmitted to the ultrasound image processor 3 for inclusion in the displayed image 17.

One way of calculating the relative position of the magnetometric detector and ultrasound probe 2 compared to the tool 5 is to use a mathematical model of the magnetic field emanating from the tool 5 and to fit the model to the magnetic field values measured by the magnetometric detector 12. This method is used in the system described in our copending International patent application no. PCT/EP2011/065420. This method works well when a mathematical model of the field can be constructed, for example as multiple monopoles in our earlier patent application, or by taking the exact geometry of the tool 5 into account and solving an integral of dipoles—aligned with the expected poles—over the geometry of the tool. If an analytical solution is too complex, then a numerical solution can be applied involving solving the integral numerically at each step in the optimisation of the fit of the measured magnetic field values. However, both the analytic and numerical methods require significant processor power and are very specific to the exact geometries being considered. For example, different surgical needles have different bevels and thicknesses, and different instruments may have different magnetic characteristics.

In this invention, to allow the magnetic tracking system to accurately track different shaped tools 5, a look-up table is provided based on pre-measured values of the magnetic field emanating from the tool of interest. The look-up table comprises an array of magnetic field values at a range of positions around the tool. The values measured by the magnetometers 120 during tracking of the tool 5 are then compared to the look-up table to find the position of the tool 5 relative to the magnetometer array 100.

For each position in space and orientation of the tool relative to the magnetometers the look-up table stores the magnetic field strength and direction at each sensor (based on previous field measurements as discussed in more detail below). In the example of a long cylindrical shaped tool such as a needle only 5 degrees of location/orientation freedom are required i.e. B_{LU}=f(k,x,y,z,θ,Φ) where k indexes the sensors, x,y,z are their location in space and θ,Φ their angular orientation compared to the tool. The data in the look-up table is preferably organized in a 6 dimensional array such that each indice in that array represents the sensor and physical spatial/orientation parameters.

Knowing the relative positioning of the magnetometers 120 in the array allows efficient searching algorithms to be used to find the corresponding values in the look-up table, for example, when trying to detect a tool, at each point in time at each sensor, values Bₖ are measured which can be compared to values in the look-up table to calculate delta:

delta=Σ(Bk−B_{LU})²=Σ(Bk−f(k,x,y,z,θ,Φ))²—summing over k sensors.

To find the correct spatial position and orientation of the tool, delta is minimized over the 5 variables in the “function” or indices in the look-up table. This is a classic optimization problem which can be solved efficiently using non-linear methods such as Levenberg-Marquardt techniques.

The relative position of the tool 5 and magnetometric detector 12 can also be interpolated from values in the look-up table, allowing a position estimate with greater accuracy than the resolution of the look-up table.

Furthermore, because the magnetic field from the tool 5 has several axes of symmetry, the size of the look-up table can be significantly reduced whilst still covering the space all around the magnetic tool. Preferably the look-up table includes magnetic field values covering a range of up to 50 mm from the longitudinal axis of the tool 5 with an accuracy of 1 mm in position and two degrees of angular freedom of the probe with an accuracy of 1 degree.

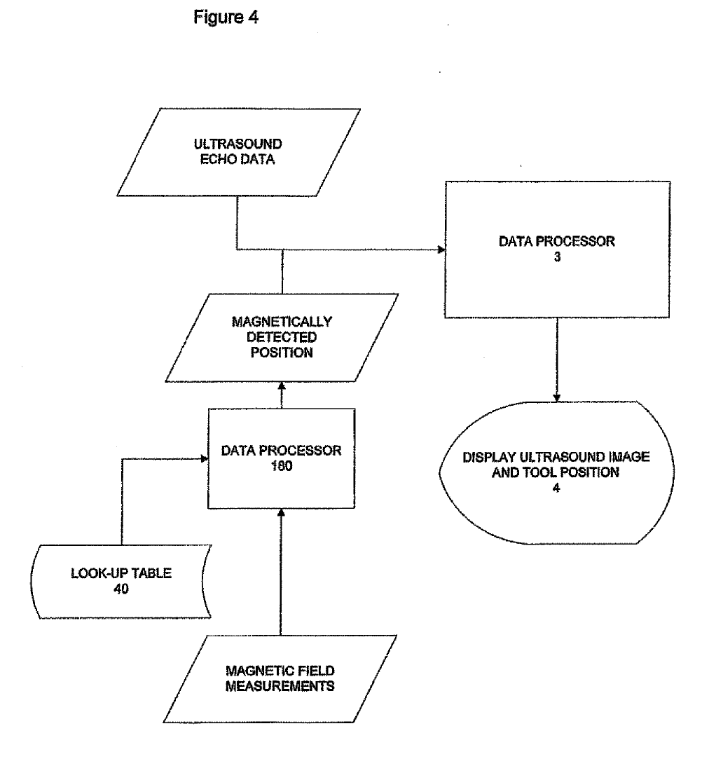

FIG. 4 illustrates the processing to produce a combined image showing the anatomy as imaged by ultrasound and the tool 5 as detected by the magnetic position detection system. As discussed above the data processor 180 in the base station 14 receives the magnetic field measurements and it compares these to stored values for the magnetic field from the tool held in a look-up table 40. By finding the closest match between the magnetic field measurements and the magnetic field values in the look-up table, the data processor can read from the look-up table the position and orientation of the tool 5, and optionally interpolate to improve accuracy. This magnetically detected position is then transmitted to the data processor 3 of the ultrasound imaging system, and the data processor 3 also receives the ultrasound echo data from the transducer probe 2. The data processor 3 processes the ultrasound echo data to produce a 2D ultrasound image which is displayed on display 4, and also uses the magnetically detected position of the tool 5 to display an image 17 of the tool overlaid on the ultrasound image.

If the tool 5 is in the imaging plane of the ultrasound transducer 2 the tool can be displayed as a solid line as illustrated schematically in FIG. 1. It is possible, however, that the tool is not in the ultrasound imaging plane. In such a case it is possible to display a position of the tool as projected onto the ultrasound image plane and to indicate in the display that it is a projected position by changing its display style. For example it can be displayed dotted and/or in a different colour. The tool is always visualised as a line, the end of which corresponds to the tool's tip. It is possible for the colour or display style to change depending upon whether the tool is in front of behind the imaging plane, and indeed if it cuts the imaging plane, parts behind can be displayed in one way and parts in front in another way.

It is also possible to display the whole expected needle track on the image display, this being a straight line extension of the tool's extent. Where anatomical features can be identified in the ultrasound image it is also possible to highlight the intersection of the needle track with these features, for example by displaying a circle or rectangle on the intersection.

Although in FIG. 1 the magnetometers 120 are displayed in an array across the front of the ultrasound transducer 2, it is also possible for them to be arranged in different ways on the ultrasound transducer 2.

Optionally the transducer 2 can also be provided with an inertial measurement unit which measures the position and orientation of the transducer by monitoring its acceleration from an initial position.

### Magnetic Tool

The magnetic tool 5 is at least partly a permanent magnet, however the tool 5 may include a magnetic component which is a non-permanent magnet, e.g. an electromagnet, e.g. a solenoid to which an electric current can be applied to create the magnetic field. Alternatively the inserted part of the tool 5 may be magnetic due to magnetic induction from outside the body or from another part of the tool 5.

The magnetisation may be provided by a magnetic coating, preferably a permanent magnetic coating. For this purpose, it may for example comprise permanent magnetic particles, more preferably nanoparticles. A “nanoparticle” is a particle that in at least two spatial dimensions is equal to or smaller than 100 nm in size.

In one embodiment of the invention, tool has an essentially uniform magnetization. In another embodiment, the magnetization is non-uniform in at least one dimension, i.e. the magnetic moment varies in magnitude and/or direction as a function of the location on the tool, thereby creating a one- or more-dimensional magnetic pattern, e.g. similar to the pattern of a conventional magnetic strip (at least one-dimensional) or disk (two-dimensional) as it is used for the storage of information e.g. on credit cards. In a preferred embodiment of the invention, a one-dimensional magnetic pattern may be recorded along the length of the tool. Advantageously, such a pattern can be useful to identify the tool, and preferably to ensure that the correct look-up table is selected for position detection. Also, by marking certain parts of the tool with different magnetic codes, these parts can be distinguished. It is an achievable advantage of this embodiment of the invention that the position and/or orientation of the tool can be better determined, as individual parts of the tool can be identified and individually tracked with respect to their position and/or orientation. In particular, advantageously, a varying shape of the tool, for example a needle bending under pressure, can be tracked. Moreover, a deformed tool and/or its deformation or degree of deformation can be determined more easily.

### Look-Up Table

The look-up table 40 stores values of the magnetic field around the tissue penetrating medical tool 5. The field and thus these values vary between types, sizes and brands of tool and so, preferably, different look-up tables are provided for different tools. Preferably the magnetic field values stored extend for a range of up to 50 mm from the centre of the tool with a position resolution of 1 mm in each of the three dimensions. The look-up table may store the values at individual spatial locations around the tool, or may store the values for each of the four magnetometric sensors 120 (or whatever number of sensors are in the array 100) for each relative location and orientation of the ultrasound probe 2 and tool 5. In this case magnetic field values are stored for the same range of up to 50 mm from the centre of the tool, in three dimensions, with a positional resolution of 1 mm, and for 2 degrees of angular freedom with an angular resolution of 1 degree. This constitutes 500,000 data points but using the symmetry of the field it can be reduced to 125,000 different data points. Although it is possible to measure the field at each of these locations in an initial measurement process, rotational transformations can be used to reduce the number of measurements required to take account of the angular degrees of freedom between the probe and tool. Given the number of measurements required and the accuracy, the measurement process to populate the look-up table with data is preferably conducted by a robotic system which moves an array of sensors corresponding to the sensors 120 around a tool while taking magnetic field measurements. This measurement process is conducted for each type of tool which it is desired to track.

In practice static background field variations i.e. stray fields resulting from nearby electronics, metal objects, or magnets can interfere with measurements causing inaccuracies. These can be removed by performing the measurements both with and without the tool present. To eliminate any temporal variation in background fields, filtering measured data over time is sometimes necessary.

Furthermore, knowing that magnetic fields vary smoothly in free space, rather than measuring at every required position with the finest required resolution, fewer, wider-spaced, measurements may be taken with the data for intermediate positions being interpolated from the measurements.

Of course if lower resolution tracking is acceptable, then the look-up table may be populated with data representing lower resolution field measurements. Also if a smaller detection space is acceptable, the size of the look-up table can be reduced correspondingly.

On occasion the subject may have more than one magnetic object in or around their body. For example there may be metallic prosthesis or other components such as bone screws which have a magnetic signature. It is straightforward to measure the magnetic fields emanating from such structures and to provide these measurements in the form of look-up tables and as magnetic fields sum straightforwardly, the tables may be combined together to allow magnetic position detection in such circumstances.

Although it was mentioned above that medical tools such as needles or catheters are not reliably visible in ultrasound images themselves, sometimes the tool 5 will be imaged by the ultrasound probe and in those circumstances the ultrasound information on the position of the tool can be combined with the magnetically detected position information to provide a better fused estimate of the tool position relative to the ultrasound probe.

1. An ultrasound imaging system for image-guided medical procedures, the system comprising:
an ultrasound transducer probe for transmitting ultrasound into a subject and receiving ultrasound echoes from the subject and outputting ultrasound echo data;
a magnetometric detector attached to the ultrasound transducer probe for detecting a magnetic field emanating from a tissue-penetrating medical tool and outputting measurements of the magnetic field;
at least one data processor adapted to receive the ultrasound echo data and process it to produce an ultrasound image and adapted to receive the magnetic field measurements and process them to determine the position of the tissue-penetrating medical tool relative to the ultrasound transducer probe; and
a data store storing a look-up table of values of the magnetic field emanating from the tissue-penetrating medical tool;
the at least one data processor being adapted to determine the position of the tissue-penetrating medical tool relative to the ultrasound transducer probe by comparing the magnetic field measurements to the values of the magnetic field stored in the look-up table.

2. The system according to claim 1, wherein the tissue-penetrating medical tool is a needle, stylet, cannula or catheter.

3. The system according to claim 1, wherein a respective plurality of look-up tables are provided for respective different tissue-penetrating medical tools.

4. The system according to claim 1, wherein the look-up table stores values of direction and magnitude of the magnetic field at a plurality of spatial positions around the tissue-penetrating medical tool.

5. The system according to claim 1, wherein the look-up table stores values of direction and magnitude of the magnetic field at the positions of each of an array of magnetometric sensors forming said magnetometric detector for a plurality of angular orientations of said array at each of a plurality of spatial transducer positions around the tissue-penetrating medical tool.

6. The system according to claim 1, wherein the look-up table stores values of the magnitude of the magnetic field in each of three orthogonal directions at each spatial position.

7. The system according to claim I, wherein the magnetometric detector comprises an array of magnetometric sensors.

8. A method comprising the steps of:
providing an ultrasound transducer probe for transmitting ultrasound into a subject and receiving ultrasound echoes from the subject and outputting ultrasound echo data;
attaching a magnetometric detector to the ultrasound transducer probe for detecting a magnetic field emanating from a tissue-penetrating medical tool and outputting measurements of the magnetic field;
providing at least one data processor adapted to receive the ultrasound echo data and process it to produce an ultrasound image and adapted to receive the magnetic field measurements and process them to determine the position of the tissue-penetrating medical tool relative to the ultrasound transducer probe; and
providing a data store storing a look-up table of values of the magnetic field emanating from the tissue-penetrating medical tool;
wherein the at least one data processor is adapted to determine the position of the tissue-penetrating medical tool relative to the ultrasound transducer probe by comparing the magnetic field measurements to the values of the magnetic field stored in the look-up table.

9. The method according to claim 8, wherein the tissue-penetrating medical tool is a needle, stylet, cannula or catheter.

10. The method according to claim 8, wherein a respective plurality of look-up tables are provided for respective different tissue-penetrating medical tools.

11. The method according to claim 8, wherein the magnetometric detector comprises an array of magnetometers.

12. The method according to claim 8, comprising the step of obtaining values for the look-up table by measuring the field emanating from a tissue-penetrating medical tool and storing the measured values in the look-up table.

13. The method according to claim 12, further comprising interpolating the measurements to produce interpolated values and storing both the measurements and the interpolated values in the look-up table.

14. The method according to claim 12, further comprising measuring magnetic field values for one region around the tissue penetrating medical tool and using symmetry to represent the magnetic field in other areas around the tissue-penetrating medical tool.

15. The method according to claim 12, wherein the magnetic field is measured robotically.

16. The method according to claim 12, wherein the magnetic field is measured using an array of multiple sensors.

17. The method according to claim 8, wherein the look-up table stores values of the direction and magnitude of the field at a plurality of spatial positions around the tissue-penetrating medical tool.

18. The method according to claim 8, wherein the look-up table stores values of the direction and magnitude of the field at the position of each of an array of magnetometric sensors forming said magnetometric detector for a plurality of angular orientations of said array at each of a plurality of spatial transducer positions around the tissue-penetrating medical tool.

19. The method according to claim 8, wherein the look-up table stores values of the magnitude of the magnetic field in each of three orthogonal directions at each spatial position.
